(19) United States
(12) Patent Application Publication
(10) Pub. No.: US 2024/0382304 A1
(43) Pub. Date: Nov. 21, 2024

(54) Low Stress Surgical Heart Valve

(71) Applicant: Krishna Raj KUPUMBATI, Irvine, CA (US)

(72) Inventor: Krishna Raj KUPUMBATI, Irvine, CA (US)

(21) Appl. No.: 18/670,056

(22) Filed: May 21, 2024

Related U.S. Application Data
(60) Provisional application No. 63/467,988, filed on May 21, 2023.

(51) Int. Cl. A61F 2/24

(52) U.S. Cl. CPC A61F 2/2418; A61F 2220/0008; A61F 2220/0075; A61F 2230/0013

(57) ABSTRACT

A surgical replacement tissue heart valve comprising three separate arcuate frame components that are connected together to form a continuous closed frame structure. A variable spacing located between each arcuate frame component reduces stress transferred to the leaflet commissures during the heart contraction cycle thereby providing improved leaflet durability.

### BACKGROUND

Replacement tissue heart valves are used by surgeons in open heart surgery to replace aortic valves, mitral valves, and other native valves of the heart. Such replacement valves often have a base ring formed of a polymeric material that is covered by a fabric that can be sewn by the physician directly to the annulus of the native valve. Attached to the base ring are typically three posts that extend in a direction downstream of the base ring. The three posts are used to provide a support for the commissures of three tissue leaflets which are attached to the posts; each of the tissue leaflets can attach in a crown-shaped path from one post and further to the base ring and still further to a second neighboring post. The posts can be constructed out of a polymeric material and can be contiguous with the base ring. Alternately, the posts can have a wire construction that provides flexibility in the radial direction; the wire construction provides for post deflection that can reduced stress at the leaflet commissures during systolic contraction of the heart.

It has been identified in explanted replacement heart valves that calcification often occurs at or near the commissures. The outward forces placed onto the commissures cannot be overcome by providing a more flexible post resulting in potential calcification and failure of the valve leaflet at or near the commissures. An increase in the flexation of the posts outwards during systole can result in central regurgitation of blood due to lack of full coaptation of the leaflets along the central axis of replacement valve.

What is needed is a replacement valve frame that will reduce the stress placed onto the commissures of the leaflet but will not cause central regurgitation of blood. The reduction in stress at or near the leaflet commissures will result in less leaflet calcification and a greater leaflet durability.

### SUMMARY

The present invention is a surgical replacement heart valve having a frame that is formed of three separate arcuate frame components, for example, (or alternately two separate frame components) connected together along a perimeter of the frame via sutures or fibers, for example. Each of the three arcuate frame components can form an arc of 120 degrees (range 100-140 degrees) such that when the frame components are connected together they form a frame that has a closed base structure. The frame can circumferentially expand and contract during systole and diastole of the heart contraction cycle. The three separate frame components allow for circumferential movement between each component resulting in reduced stress at or near the commissures of replacement leaflets that are attached to the three frame components.

The frame has a ring-like base that can be formed of a polymeric material that retains a generally arcuate shape such that three individual base segments can be connected together via sutures or elastomeric fibers, for example, to form a circular shape for an aortic replacement valve or form a “D-shape” for a mitral replacement valve, for example. The three individual base sections are able to extend circumferentially at connection sites to allows the perimeter of the base to enlarge and contract during heart contraction and relaxation function. The frame base forms a sealing attachment to an upstream portion of the replacement leaflet. The frame base can be covered by a sewing ring fabric that allows the physician to attach the frame to the native annulus via sutures, for example.

Attached to each end of an individual base section are two post segments, one at each end of a base section. When two neighboring frame sections are connected together two neighboring post segments approximate each other to form a post. The post segments are connected together via sutures or elastomeric fiber, for example, and allow the post segments to elastically separate from each other in a circumferential direction and return during heart contraction and relaxation. This circumferential movement and flexibility between two post segments that form a single post will reduce the stresses at the leaflet commissures that are attached to the posts.

Each replacement leaflets can be attached along a crown-shaped attachment path to the two post sections of a base section and to a base section of a frame component. The frame can have three frame components for a replacement valve having three replacement leaflets, or the frame can have two frame components, each of which is attached to a separate leaflet. The free edges of the leaflets coapt together at the downstream end of the replacement heart valve to direct blood flow downstream and prevent blood flow in an upstream direction.

A covering material such as a woven or knitted fabric such as Dacron or polyurethane, for example, can be attached to two neighboring post segments and between two neighboring base sections to prevent blood leakage through the spacing between such post segments or base sections.

### BRIEF DESCRIPTION OF THE DRAWINGS

FIG. 1A is a perspective view of a frame comprising three frame components that are connected together to form a closed base structure.

FIG. 1B is a perspective view of a frame component.

FIG. 1C is a perspective view of a frame component with a wire post construction.

FIG. 1D is a perspective view of a post segment showing a post connection fiber extending within a channel of a post segment.

FIG. 2A is a spayed-out view of three leaflets showing crown-shaped leaflet edges and leaflet free edges.

FIG. 2B is a perspective view of a frame having three leaflets attached to the frame along a crown-shaped attachment path.

FIG. 3 is a perspective view of a frame having a sewing ring attached along the base of the frame.

FIG. 4A is a perspective view of a frame comprising two frame components wherein one frame component has a flattened shape in the circumferential direction.

FIG. 4B is a top view of a valve frame having a base section with a flattened shape.

### DETAILED DESCRIPTION

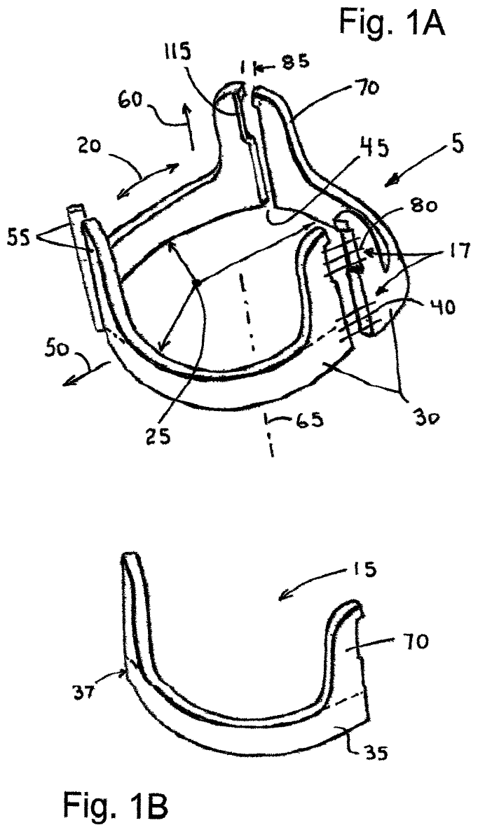

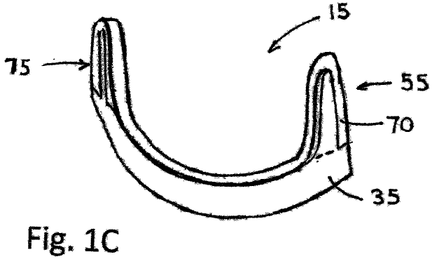

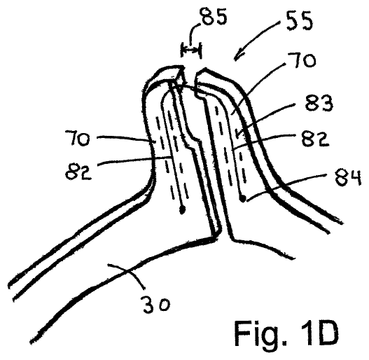

FIGS. 1A, 1B, 1C, 1D, 2A, 2B, and 3 show a perspective view of an embodiment of the valve frame 5 and replacement valve 10 of the present invention having three individual arcuate frame components 15 that are connected together in a circumferential direction 20. Each of the arcuate frame components 15 extend around the circumference of the valve frame 5 by 120 degrees (range 100 degrees to 140 degrees). Each frame component 15 comprises a base section 35 and two post segments 70 located at each end of each frame component. It is understood that two individual frame components 15 can also be connected together to form the valve frame 5 of the present invention. The three arcuate frame components 15 form a closed frame structure 25 after the frame components 15 have been connected together via frame component connections 17. The frame component connections 17 can be formed from sutures, fibers, adhesives, or other connection means or methods that provide a flexible connection between two neighboring frame components 15.

As seen in FIG. 1A, the base 30 (the circumferentially arcuate portions valve frame 5) is formed from three base sections 35 that are each connected at each base section end 37 to a neighboring base section 35 via base connections 40 formed from sutures, polymeric or metallic fibers, or other connection material or method that allows for elastic separation, return of a base variable spacing 45, and flexibility between neighboring base sections 35. A base variable spacing 45 of 1 mm (range 0-3 mm) in a circumferential direction 20 is found between neighboring base sections 35 and can vary due to radially outward 50 and inward forces during systole and diastole. The base sections 35 can be formed from a polymeric material such as Delrin, polyether ester ketone (PEEK), or other polymeric material, metal, composite, or material that is implantable and can retain an arcuate shape that can form a circular or curved shape for the base 30 with all three base sections 35 connected together.

Attached to the base sections 35 are three posts 55 extending in a downstream direction 60 parallel to the frame central axis 65. Each post 55 is formed of two post segments 70; each post segment 70 of a single post 55 is attached to a post segment 70 of a neighboring base section 35 to form a post 55. The post segment 70 can be formed from the same material as the base section 35 and can be contiguous with the base section 35. Alternately, the post 55 can be formed from a wire construction 75 including a metal or polymeric fiber or expanded stent-like structure that can deflect in a radial direction during heart valve functioning as shown in FIG. 1C. The post segment 70 can be bonded to the base section 35 via adhesives, thermal bonding, solvent bonding, mechanical fit, or via other bonding means and via other methods. The post segments 70 of a single post 55 can be connected together to form a post connection 80 via sutures, fibers, elastic materials, or other connection materials that allow for separation and return of the post segments 70 during systole and diastole. A post variable spacing 85 in a circumferential direction 20 is found between two post segments 70 of a single post 55; the post variable spacing 85 is 1 mm (range 0-3 mm) and is due to radially outward 50 and inward forces that occur during systole and diastole. The post variable spacing 85 and base variable spacing 45 allows for circumferential movement between post segments 70 of a post 55 and between two neighboring base sections 35 resulting in a reduction in stress at or near the commissures 90 of replacement leaflets 95 thereby providing a greater durability to the replacement leaflets 95.

The post connection 80 can be formed from one or more post connection fibers 82 that extend from a fiber attachment 84 on one post segment 70 to a fiber attachment 84 on a neighboring post segment 70 of a single post 55 as shown in FIG. 1D. The post connection fiber 82 can have an elastic capability that allows for movement and separation of one post segment 70 relative to a neighboring post segment 70 forming the post variable spacing 85; this post variable spacing changes from systole to diastole and provides the valve frame of the present invention with a reduced stress onto the commissures of the replacement leaflets. The post connection fiber 82 can be contained within and extend along a post segment channel 83 located within one or both post segments 70 as shown in FIG. 1D. The post segment channel 83 can provide an increased length for the post connection fiber 82 and can provide an increased amount of post variable spacing 85 that is provided during systole and diastole.

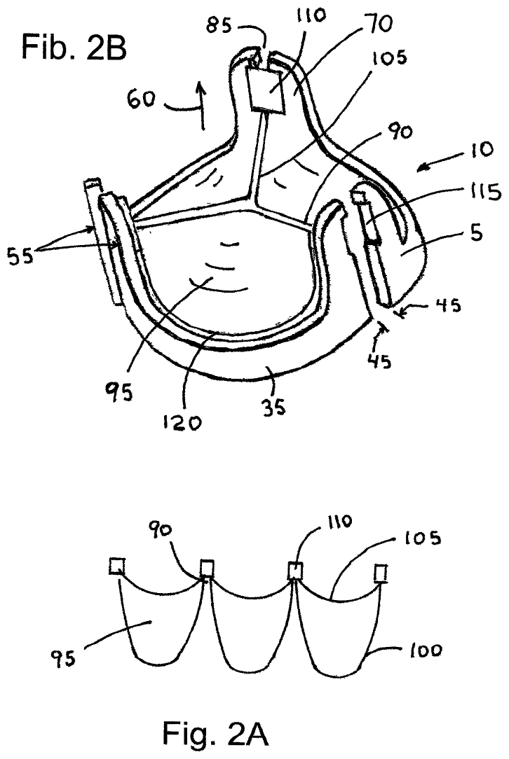

FIGS. 2A and 2B show three replacement leaflets 95 that have been attached to the valve frame 5 of an embodiment of the replacement valve of the present invention. The replacement leaflets 95 can be formed from tissue, cellular structure, extracellular matrix, or polymeric material, or composite material that can be formed into a thin membrane capable of holding stresses found in a functioning heart valve. The replacement leaflets 95 can be formed with a leaflet attachment edge 100 and a free edge 105 as shown in FIG. 2A. The leaflet attachment edge 100 which is attached to the frame component along a frame component attachment path 120 can be a crown-shaped leaflet attachment edge 100. A commissural pad 110 located between two neighboring leaflets provides a location and material by which the commissures 90 of the replacement leaflets 95 can be attached to the commissure attachment site 115 located on each post 55 as shown in FIG. 2B. The commissural pad 110 can be formed from a material or fabric that can provide movement or stretch to accommodate the post variable spacing 85; such materials include Dacron fabric, Elgiloy materials, polyurethane, composite fabrics and materials, and materials used to form the replacement leaflets 95. A single commissural pad 110 is attached to both post segments 70 of a post 55 such that stresses onto the commissures 90 generated during systole and diastole are absorbed by the post variable spacing 85 and by the commissural pad 110, and the stresses are not transferred to the commissures 90 of the leaflets.

Each replacement leaflet 95 is also attached to the post segments 70 and base section 35 of a single frame component 15 along a frame component attachment path 120 as shown in FIG. 2B; the frame component attachment path 120 can be a crown-shaped attachment path. The replacement leaflet 95 can be attached to the frame component 15 via sutures or fibers that can withstand the forces exerted by blood pressure found within a functioning replacement valve 10. The leaflet free edges 105 located on the downstream end of the replacement valve 10 are adapted to coapt with each other to prevent blood flow in an upstream direction and provide blood flow in a downstream direction 60.

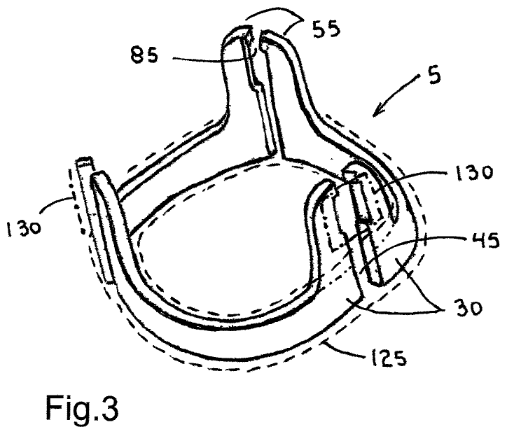

As shown in FIG. 3 a sewing ring 125 formed from a fabric material is wrapped around and attached to the base 30 of the valve frame 5 to provide a material that can be sutured by the surgeon to the native annulus of the heart to form a leak-tight seal with the annulus and prevent migration of the replacement valve 10. The sewing ring 125 can be formed from Dacron fabric, a thin polymeric material, metallic fibers, a composite fabric or other material that can be sewn to a heart annulus and hold the stresses imposed by the functioning replacement valve. A fabric cover 130 can also be placed over the base variable spacing 45 and post variable spacing 85 and attached to the post 55 and frame base 30 to prevent blood leakage through the post variable spacing 85 or base variable spacing 45 that could lead to blood regurgitation and negative sequalae.

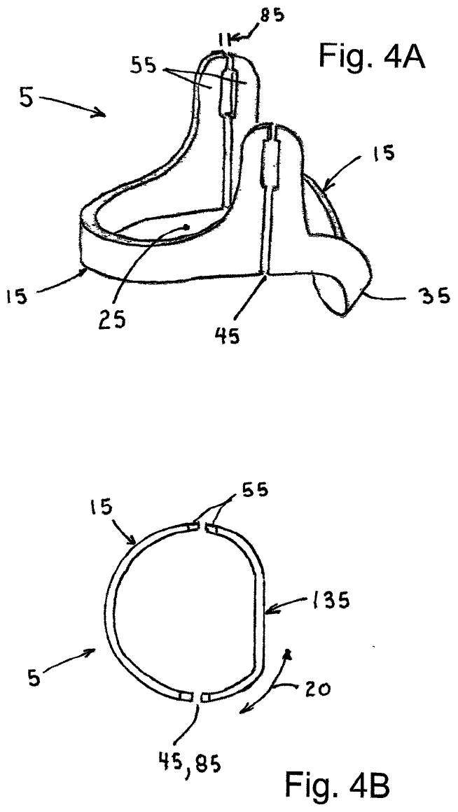

The replacement valve 10 of the present invention can have a valve frame 5 formed from two frame components 15 as shown in FIGS. 4A and 4B. The two frame components 15 can be formed from materials that are the same as those described for a frame formed from three components. To accommodate the D-shaped annulus of the mitral valve, one of the frame components 15 can have a more flattened shape 135 in a circumferential direction 20 as seen in FIG. 4B from above. The valve frame thus forms a closed base structure 25 having a D-shape. The embodiment with two frame components 15 similarly has post variable spacing 85 and base variable spacing 45 as described earlier for the embodiment having three frame components 15. Attachment of replacement leaflets 95 and commissure pad 110 to the frame is the same for this embodiment as described earlier. The attachment of a sewing ring 125 and a fabric cover 130 is also similar for this embodiment with two components as described earlier for the embodiment having three components.

Reference numerals that describe structure found in any figure in this specification can equally describe structure found in any other figure found in the specification.

1. A replacement heart valve for direct implant by a surgeon into an annulus of a native heart valve to direct blood flow in a downstream direction, said replacement heart valve comprising;
A. two or more frame components having an arcuate shape and being connected together via frame component connections in a circumferential direction to form a valve frame that forms a closed frame structure extending around a perimeter of said valve frame,
B. said frame component connections having a flexibility to provide for enlargement and reduction of said perimeter of said valve frame during systolic and diastolic movement of a heart,
C. said replacement heart valve further comprising two or more replacement leaflets, each of said two or more replacement leaflets having a commissure attached via a commissural attachment to two neighboring of said frame components.

2. The replacement heart valve of claim 1 wherein each of said two or more replacement leaflets are further attached along a leaflet attachment edge to each of said two or more frame components along a frame component attachment path.

3. The replacement heart valve of claim 2 wherein said frame component attachment path is a crown-shaped attachment path.

4. The replacement heart valve of claim 1 wherein each of said two or more frame components is comprised of a base section extending in said circumferential direction with said arcuate shape and two post segments attached to each end of said base section and extending in a downstream direction.

5. The replacement heart valve of claim 4 wherein said commissure is attached to one of said two post segments from each of said two neighboring of said frame components.

6. The replacement heart valve of claim 4 wherein said base section is formed from a polymeric material.

7. The replacement heart valve of claim 4 wherein said base section and said two post segments are formed from the same material and are contiguous with each other.

8. The replacement heart valve of claim 4 wherein said two post segments are formed from a wire construction or from an expanded stent-like structure.

9. The replacement heart valve of claim 1 wherein each of said two or more frame components form an arc of 120 degrees.

10. The replacement heart valve of claim 4 wherein said base section has a sewing ring attached thereto, said sewing ring providing said valve frame to be attached via a surgeon to the annulus via sutures.

11. The replacement heart valve of claim 4 wherein one of said two post segments from each of said two neighboring frame components have a variable spacing therebetween that can enlarge and reduce said perimeter during said systolic and diastolic movement of a heart.

12. The replacement heart valve of claim 11 wherein one of said two post segments from each of said two neighboring frame components are connected by one or more post connection fibers.

13. The replacement heart valve of claim 12 wherein said one or more post connection fibers are contained within a channel and extend along one of said post segments.

14. The replacement heart valve of claim 11 wherein one of said two post segments of each of said two neighboring frame components has a covering attached to prevent blood leakage therethrough.

15. The replacement heart valve of claim 1 wherein said two or more frame components comprise two frame components.

16. The replacement heart valve of claim 1 wherein said two or more frame components comprise three frame components.

17. The replacement heart valve of claim 1 wherein said replacement leaflets are tissue leaflets.

18. The replacement heart valve of claim 1 wherein said replacement leaflets are polymeric or composite leaflets.

19. A replacement heart valve for direct implant by a surgeon onto the annulus of a native heart valve comprising:
A. a valve frame comprised of two or more individual frame components forming a frame component connection to a neighboring frame component along a perimeter of said valve frame, said frame component connection being configured to alter said perimeter of said valve frame due to flexibility of said frame component connection during a heart contraction cycle,
B. each of said frame components comprising a base section that extends circumferentially along said perimeter, and further comprising a post segment extending downstream of said base section and attached to said base section,
C. said valve frame forming a leaflet attachment to two or more tissue or polymeric leaflets, each of said two or more tissue or polymeric leaflets having a commissure attached to one of said post segments of one of said frame components and also attached to a another of said post segments of a neighboring of said frame components.
F. whereby said commissure has reduced stress during the heart contraction cycle due to said flexibility of said frame component connection between each of said two or more frame components.

20. A replacement heart valve for direct implant by a surgeon into the annulus of a native heart valve comprising:
A. a valve frame comprised of two or more frame components connected in a circumferential direction to a neighboring frame component along a perimeter of said valve frame,
B. each of said frame components comprised of two post segments joined to a base section, said post segments extending downstream of said base sections,
C. one of said two post segments from one frame component forming a frame component connection to one of said two post segments from said neighboring frame component,
D. said valve frame forming a crown-shaped attachment of two or more tissue or polymeric leaflets to said valve frame, each of said two or more tissue or polymeric leaflets having a commissure attached via a commissural attachment to one of said post segments of one of said frame components and also attached to a another of said post segments of a neighboring of said frame components.
F. whereby said commissure has reduced stress during valvular movement of said replacement heart valve due to said commissural attachment of each of said commissure to two post segments.
